(19) United States
(12) Patent Application Publication
(10) Pub. No.: US 2024/0051120 A1
(43) Pub. Date: Feb. 15, 2024

(54) REAR-END TRANSMISSION DEVICE, SURGICAL INSTRUMENT, AND SURGICAL ROBOT

(71) Applicant: CORNERSTONE TECHNOLOGY (SHENZHEN) LIMITED, Shenzhen (CN)

(72) Inventor: Hoiwut YIP, Shenzhen (CN)

(21) Appl. No.: 18/496,620

(22) Filed: Oct. 27, 2023

Related U.S. Application Data
(63) Continuation of application No. PCT/CN2022/078980, filed on Mar. 3, 2022.

(30) Foreign Application Priority Data
Apr. 30, 2021 (CN) 202110484481.8

(51) Int. Cl. B25J 9/10; A61B 34/30

(52) U.S. Cl. CPC B25J 9/104; A61B 34/30; A61B 2034/305

(57) ABSTRACT

A rear-end transmission device, a surgical instrument, and a surgical robot are provided. The rear-end transmission device includes a first rotating member, a second rotating member, a third rotating member, a first transmission assembly, and a second transmission assembly. The first transmission assembly is connected to a first cable and a second cable and connected to the first rotating member and the third rotating member. The second transmission assembly is connected to the third cable and the fourth cable and connected to the second rotating member and the third rotating member. The first rotating member is rotatable to pull in one of the first cable and the second cable while releasing another of the first cable and the second cable.

### CROSS REFERENCE TO RELATED APPLICATIONS

This application is a continuation of PCT Patent Application No. PCT/CN2022/078980, filed Mar. 3, 2022, which claims priority to Chinese Patent Application No. CN202110484481.8, filed on Apr. 30, 2021, each of which is hereby incorporated by reference in its entirety.

### TECHNICAL FIELD

The various embodiments described in this document relate in general to the technical field of medical devices, and more specifically to a rear-end transmission device, a surgical instrument, and a surgical robot.

### BACKGROUND

In robot-assisted minimally invasive surgery, surgical instruments connected at the end of the robot enter the human body through wounds on the surface of the human body or natural canals of the human body, to be operated on tissues in the human body. The surgical instrument mainly includes an effector or tool (such as a pair of surgical forceps, a cutting tool, or a cauterizing tool) mounted to a wrist mechanism at a front end of the instrument, the wrist mechanism providing multiple degrees of freedom for movement of the front end, a main shaft extending from a rear end of the instrument to the front end, and a power and transmission device at the rear end of the instrument. The effector at the front end and the wrist mechanism are generally driven by multiple cables fixed thereto, and the cables run through the main shaft of the surgical instrument and are driven by the power and transmission device at the rear end.

For surgical forceps and other holding or cutting tools, the wrist mechanism is generally required to realize three degrees of freedom: pitch, yaw, and grip. Cooperated with extra degrees of freedom of the rear end of the robot, the wrist mechanism can realize the movement required to perform surgical operations. According to specific implementation of the wrist mechanism, the number of driving cables required for the wrist mechanism varies, for example, there are generally 4 or 6 cables configured for the wrist mechanism. In addition, in order to realize a large-scale movement (for example, rotation of −90° to 90°) of each joint of the wrist mechanism, it is necessary to arrange extra pulleys on the wrist mechanism to guide cables. However, addition of the extra pulleys may hinder the miniaturization of the front end of the surgical instrument, and the use of more cables may also increase the size and cost of the instrument.

The existing rear-end transmission device for driving the 4-cable wrist mechanism realizes release of two cables and pulling in of other two cables through swing of a connecting rod or a rocker arm, thereby realizing the pitch of the wrist mechanism. In this way, with the change of a pitch angle, a length of a pulled-in cable is not equal to a length of a released cable, which may lead to a change in tension of the cables, and then lead to problems such as reduced accuracy due to transmission error caused by loosened cables, or accelerated wear due to excessive frictional resistance caused by over-tensioned cables. To avoid such problems, one method is to limit a range of a rotation angle of a pitch joint, but this method may affect the function of the instrument. In addition, when using this mechanism, a rotation angle of an output terminal of the motor at the rear end has a nonlinear relationship with the pitch angle of the wrist mechanism, which requires extra calibration to achieve accurate position control and thus increases extra workload.

### SUMMARY

A series of simplified concepts have been introduced in this section, which will be further elaborated in the detailed description. The section of the disclosure does not mean attempting to limit key features and essential technical features of the claimed technical solution, nor does it mean attempting to determine the scope of protection of the claimed technical solution.

Embodiments of the disclosure provide a rear-end transmission device and includes a first rotating member, a second rotating member, a third rotating member, a first transmission assembly, and a second transmission assembly. The first transmission assembly is connected to a first cable and a second cable and further connected to the first rotating member and the third rotating member. The second transmission assembly is connected to a third cable and a fourth cable and further connected to the second rotating member and the third rotating member. The first rotating member is rotatable to pull in one of the first cable and the second cable and to release another of the first cable and the second cable concurrently via the first transmission assembly. The second rotating member is rotatable to pull in one of the third cable and the fourth cable and to release another of the third cable and the fourth cable concurrently via the second transmission assembly. The third rotating member is rotatable to perform via the first transmission assembly and the second transmission assembly: pulling in at least one of the first cable and the second cable while releasing at least one of the third cable and the fourth cable; or pulling in at least one of the third cable and the fourth cable while releasing at least one of the first cable and the second cable.

According to an embodiment of the present disclosure, the rear-end transmission device is connected to four driving cables of the wrist mechanism, such that the wrist mechanism works cooperatively with the rear-end transmission device to achieve pitch, yaw, and grip of the wrist mechanism by pulling in or releasing the driving cables (i.e., the first cable, the second cable, the third cable, and the fourth cable), which is simple in structure and accurate in transmission. In addition, a rotation angle of the rotating member is linearly related to a pitch angle, a yaw angle, or a grip angle of the wrist mechanism, and therefore, equal-length release and/or pulling-in of the driving cables can be ensured even when the above-mentioned angles of the wrist mechanism vary in a wide range.

In some embodiments, the first rotating member is rotatable around a first central axis of the first rotating member, the second rotating member is rotatable around a second central axis of the second rotating member, and the third rotating member is rotatable around a third central axis of the third rotating member; and the first central axis is substantially parallel to the second central axis and substantially parallel to the third central axis.

In some embodiments, the first transmission assembly includes a first movable pulley, a first transmission cable, a second movable pulley, and a second transmission cable. The first movable pulley is connected to the first cable. The first transmission cable rides on the first movable pulley, where the first transmission cable has one end connected to the first rotating member and has another end connected to the third rotating member. The second movable pulley is rotatable around a second shaft and connected to the second cable. The second transmission cable rides on the second movable pulley, where the second transmission cable has one end connected to the first rotating member and has another end connected to the third rotating member. The first transmission cable wraps around the first rotating member in a direction opposite to a direction in which the second transmission cable wraps around the first rotating member, and the first transmission cable wraps around the third rotating member in a direction same as a direction in which the second transmission cable wraps around the third rotating member. In rotation of the first rotating member or the third rotating member, the first movable pulley is driven to move by the first transmission cable and the second movable pulley is driven to move by the second transmission cable.

In some embodiments, the first transmission assembly further includes a first guide pulley, where a portion of the first transmission cable located between the first rotating member and the first movable pulley rides on the first guide pulley, such that a portion of the first transmission cable located between the first guide pulley and the first movable pulley is substantially parallel to a portion of the first transmission cable located between the first movable pulley and the third rotating member.

In some embodiments, a portion of the second transmission cable located between the first rotating member and the second movable pulley is substantially parallel to a portion of the second transmission cable located between the second movable pulley and the third rotating member and substantially parallel to the portion of the first transmission cable located between the first movable pulley and the third rotating member.

In some embodiments, the second transmission assembly includes a third movable pulley, a third transmission cable, a fourth movable pulley, and a fourth transmission cable. The third movable pulley is connected to the third cable. The third transmission cable rides on the third movable pulley, where the third transmission cable has one end connected to the second rotating member and has another end connected to the third rotating member. The fourth movable pulley is rotatable around a fourth shaft and connected to the fourth cable. The fourth transmission cable rides on the fourth movable pulley, where the fourth transmission cable has one end connected to the second rotating member and has another end connected to the third rotating member. The third transmission cable wraps around the second rotating member in a direction opposite to a direction in which the fourth transmission cable wraps around the second rotating member, and each of the third transmission cable and the fourth transmission cable wraps around the third rotating member in a direction opposite to a direction in which each of the first transmission cable and the second transmission cable wraps around the third rotating member. In rotation of the second rotating member or the third rotating member, the third movable pulley is driven to move by the third transmission cable and the fourth movable pulley is driven to move by the fourth transmission cable.

In some embodiments, the first movable pulley is pivotably connected to a first pulley seat via a first shaft, and the first pulley seat is connected to the first cable, where the second movable pulley is pivotably connected to a second pulley seat via a second shaft, and the second pulley seat is connected to the second cable, where the first shaft is substantially parallel to the second shaft and substantially parallel to the first central axis; and/or the third movable pulley is pivotably connected to a third pulley seat via a third shaft, and the third pulley seat is connected to the third cable, where the fourth movable pulley is pivotably connected to a fourth pulley seat via a fourth shaft, and the fourth pulley seat is connected to the fourth cable, where the third shaft is substantially parallel to the fourth shaft and substantially parallel to the first central axis.

In some embodiments, the second transmission assembly further includes a second guide pulley, where a portion of the fourth transmission cable located between the second rotating member and the fourth movable pulley rides on the second guide pulley, such that a portion of the fourth transmission cable located between the second guide pulley and the fourth movable pulley is substantially parallel to a portion of the fourth transmission cable located between the fourth movable pulley and the third rotating member.

In some embodiments, a portion of the third transmission cable located between the second rotating member and the third movable pulley is substantially parallel to a portion of the third transmission cable located between the third movable pulley and the third rotating member, and substantially parallel to the portion of the fourth transmission cable located between the fourth movable pulley and the third rotating member.

Embodiments of the disclosure further provide a surgical instrument including the rear-end transmission device described in any embodiment of the disclosure.

Embodiments of the disclosure further provide a surgical robot including the surgical instrument described in any embodiment of the disclosure.

### BRIEF DESCRIPTION OF THE DRAWINGS

The following drawings of the disclosure are herein used as a part of the disclosure for understanding the disclosure. Embodiments of the present disclosure are described with reference to the accompanying drawings to explain the principles of the present disclosure, in which:

FIG. 1 is a schematic structural view of a surgical robot according to embodiments of the present disclosure;

FIG. 2 is a schematic partial structural view of the surgical robot of FIG. 1 including a wrist mechanism, a first cable, a second cable, a third cable, and a fourth cable;

FIG. 3 is another schematic partial structural view of the surgical robot of FIG. 1 including a rear-end transmission device, a first cable, a second cable, a third cable, and a fourth cable; and

FIG. 4 is a schematic partial structural view of the rear-end transmission device of the surgical robot in FIG. 1.

### DETAILED DESCRIPTION OF THE EMBODIMENTS

Detailed illustration is given in the following description to provide a more thorough understanding of the disclosure. However, it will be apparent to those skilled in the art that embodiments of the present disclosure may be practiced without one or more of these details. In other examples, some technical features well known in the art are not described in order to avoid confusion with embodiments of the present disclosure.

In order to fully understand the embodiments of the present disclosure, detailed structures will be set forth in the following description. Apparently, the implementation of the embodiments of the present disclosure is not limited to particular details familiar to those skilled in the art. It is to be noted that ordinal numbers such as “first” and “second” referenced in the disclosure are merely identifiers and do not have any other meaning, e.g., does not represent a specific order. In addition, for example, the term “first component/member” does not imply the existence of a “second component/member”, and the term “second component/member” does not imply the existence of the “first component/member”. The terms “up”, “down”, “front”, “back”, “left”, “right”, and similar expressions used in the disclosure are merely intended to explain the disclosure rather than limit the disclosure.

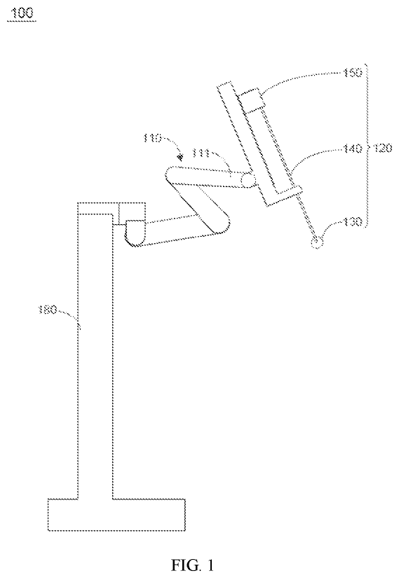

As shown in FIG. 1, embodiments of the present disclosure provide a rear-end transmission device 150, a surgical instrument 120, and a surgical robot 100. The surgical robot 100 includes a movable robotic arm 110 and a surgical instrument 120 mounted to the robotic arm 110. The surgical instrument 120 includes the rear-end transmission device 150. As known in the art, the surgical instrument 120 can achieve a number of functions, and includes but is not limited to a pair of surgical forceps or grippers of different shapes and sizes, a needle driver, a pair of scissors, or a cauterizing tool.

As shown in FIG. 1, the surgical robot 100 mainly includes a base 180, at least one robotic arm 110 rotatably disposed at an upper end of the base 180, and at least one surgical instrument 120. Each surgical instrument 120 is mounted to a port of a respective robotic arm 110. Merely one robotic arm 110 is exemplarily shown in FIG. 1, the robotic arm 110 includes a plurality of links 111 connected sequentially, and each of the plurality of links 111 is rotatable. In a case where the surgical robot 100 includes multiple robotic arms 110, each robotic arm 110 is provided with a respective surgical instrument 120 which is mounted to a port of the robotic arm 110. Each surgical instrument 120 is detachably coupled to the respective robotic arm 110, to facilitate replacement or repairment of the surgical instrument 120. Therefore, the surgical instrument 120 mounted to the respective robotic arm 110 may be used for a particular medical procedure or may be changed during the medical procedure to provide a desired clinical function.

The robotic arm 110 includes at least one docking port. The at least one docking port generally includes an output of a driving motor configured to provide mechanical power for operation of the surgical instrument 120. The at least one docking port may further include an electrical interface to which the surgical instrument 120 is coupled, to identify a type of a device coupled to the docking port and to obtain parameters of the device.

The surgical instrument 120 generally includes the rear-end transmission device 150, a main shaft 140 extending from the rear-end transmission device 150, and a wrist mechanism 130 at a distal end of the main shaft 140. Driving cables (specifically including a first cable 141, a second cable 142, a third cable 143, and a fourth cable 144) and an electrical conductor that are connected to the wrist mechanism 130 can run through the main shaft 140 and are connected to the rear-end transmission device 150. The rear-end transmission device 150 is configured to provide mechanical coupling between the above-described driving cables and a motor driving shaft of the driving motor, so as to operate the wrist mechanism 130 by controlling movement and tension of the driving cables. The main shaft 140 is hollow and may be rigid or flexible.

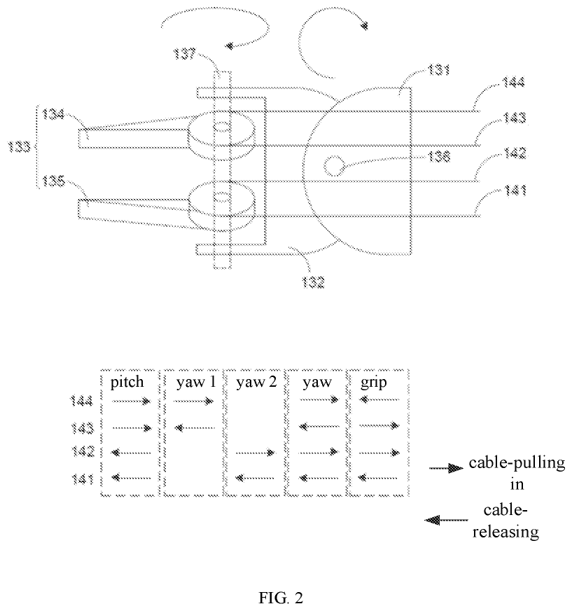

As shown in FIG. 2, the wrist mechanism 130 includes a proximal clevis 131, a distal clevis 132, and an effector 133. The distal clevis 132 is pivotably connected to the proximal clevis 131 via a first bolt 136, such that pitch of the wrist mechanism 130 is realized through rotation of the distal clevis 132 relative to the proximal clevis 131. The effector 133 includes an upper claw 134 and a lower claw 135. Each of the upper claw 134 and the lower claw 135 is pivotably connected to the distal clevis 132 via a second bolt 137, such that grip and yaw of the wrist mechanism 130 are realized through rotation of the claws 134, 135 relative to the distal clevis 132. In some embodiments, the second bolt 137 is substantially perpendicular to the first bolt 136.

The first cable 141 and the second cable 142 are wound on and connected to the lower claw 135 of the effector 133. That is, the first cable 141 and the second cable 142 may be formed integrally from one continuous cable wrapped around the lower claw 135. The third cable 143 and the fourth cable 144 are wound on and connected to the upper claw 134 of the effector 133. That is, the third cable 143 and the fourth cable 144 may be formed integrally from one continuous cable wrapped around the upper claw 134. The first cable 141, the second cable 142, the third cable 143, and the fourth cable 144 are extended along hard surfaces of guide passages (not shown) defined by the effector 133, the distal clevis 132, and the proximal clevis 131, and then reach the rear-end transmission device 150 along the main shaft 140. The guide passage may be a groove having a U-shaped or semicircular cross section.

Continuing with reference to FIG. 2, by pulling in the third cable 143 and the fourth cable 144 in equal lengths while releasing the first cable 141 and the second cable 142 in equal lengths, the distal clevis 132 is rotated clockwise around the first bolt 136 relative to the proximal clevis 131, to achieve pitch of the wrist mechanism 130 in a direction (referring to FIG. 2). Similarly, by pulling in the first cable 141 and the second cable 142 in equal lengths while releasing the third cable 143 and the fourth cable 144 in equal lengths, the distal clevis 132 is rotated counterclockwise around the first bolt 136 relative to the proximal clevis 131, to achieve pitch of the wrist mechanism 130 in a reverse direction.

The upper claw 134 is rotated clockwise around the second bolt 137 relative to the distal clevis 132 by pulling in the fourth cable 144 and releasing the third cable 143 concurrently in equal lengths (referring to yaw 1 in FIG. 2). Similarly, the upper claw 134 is rotated counterclockwise around the second bolt 137 relative to the distal clevis 132 by pulling in the third cable 143 and releasing the fourth cable 144 concurrently in equal lengths. The lower claw 135 is rotated clockwise around the second bolt 137 relative to the distal clevis 132 by pulling in the second cable 142 and releasing the first cable 141 concurrently in equal lengths (referring to yaw 2 in FIG. 2). Similarly, the lower claw 135 is rotated counterclockwise around the second bolt 137 relative to the distal clevis 132 by pulling in the first cable 141 and releasing the second cable 142 concurrently in equal lengths. The effector 133 can achieve yaw and grip through aggregate motion of the upper claw 134 and the lower claw 135, which is described in detail below.

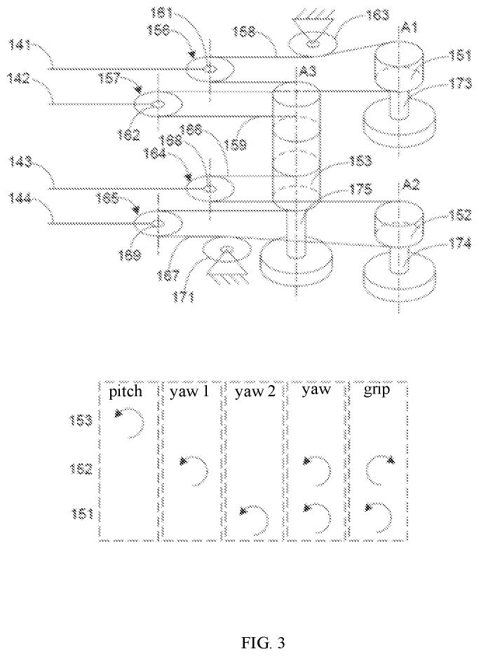

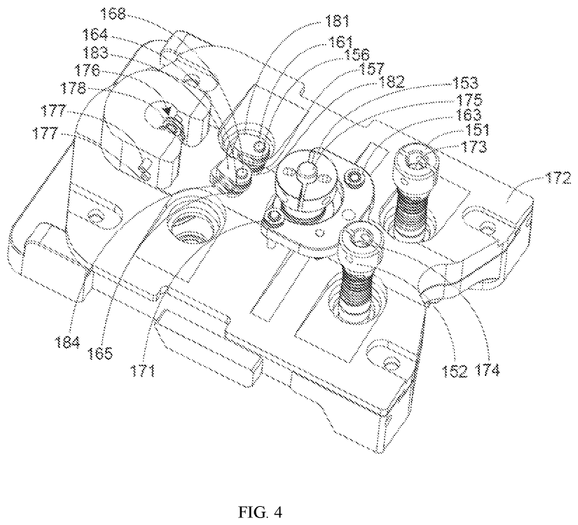

As shown in FIGS. 3 and 4, the rear-end transmission device 150 mainly includes a first rotating member 151, a second rotating member 152, a third rotating member 153, a first transmission assembly, a second transmission assembly, a first rotating shaft 173, a second rotating shaft 174, a third rotating shaft 175, and a chassis 172. Each of the first rotating shaft 173, the second rotating shaft 174, and the third rotating shaft 175 is rotatably mounted to the chassis 172, and connected with a respective driving motor. The first transmission assembly and the second transmission assembly are each mounted to the chassis 172. The chassis 172 has a plurality of interfaces and each of the plurality of interfaces corresponds to one driving motor, such that each of the first rotating shaft 173, the second rotating shaft 174, and the third rotating shaft 175 can be stably connected with the corresponding driving motor when the surgical instrument 120 is properly mounted, so as to realize the transmission of rotational motion.

The surgical robot 100 may include at least three driving motors. The three driving motors are connected to the first rotating shaft 173, the second rotating shaft 174, and the third rotating shaft 175, respectively. The first rotating shaft 173, the second rotating shaft 174, and the third rotating shaft 175 are arranged substantially parallel to each other. The first rotating shaft 173, the second rotating shaft 174, and the third rotating shaft 175 are arranged at three tips of a substantial triangle, respectively. It shall be understood that there is no restriction on arrangement of the first rotating shaft 173, the second rotating shaft 174, and the third rotating shaft 175. The first rotating shaft 173, the second rotating shaft 174, and the third rotating shaft 175 may also be arranged in a straight line as required. The first rotating shaft 173 is fixedly connected to the first rotating member 151 to drive rotation of the first rotating member 151. The second rotating shaft 174 is fixedly connected to the second rotating member 152 to drive rotation of the second rotating member 152. The third rotating shaft 175 is fixedly connected to the third rotating member 153, to drive rotation of the third rotating member 153. In embodiments of the disclosure, each of the first rotating member 151, the second rotating member 152, and the third rotating member 153 is a winch.

The first transmission assembly is connected to the first cable 141 and the second cable 142 and further connected to the first rotating member 151 and the third rotating member 153. The first transmission assembly mainly includes a first movable pulley 156, a second movable pulley 157, a first transmission cable 158, and a second transmission cable 159. The first movable pulley 156 is pivotably connected to the first pulley seat 181 via the first shaft 161, the first movable pulley 156 is pivotable around the first shaft 161, and the first cable 141 is connected to the first pulley seat 181. The second movable pulley 157 is pivotably connected to the second pulley seat 182 via the second shaft 162, the second movable pulley 157 is pivotable around the second shaft 162, and the second cable 142 is connected to the second pulley seat 182.

The first transmission cable 158 rides on the first movable pulley 156. The first transmission cable 158 has one end connected to the first rotating member 151 and has the other end connected to the third rotating member 153. The second transmission cable 159 rides on the second movable pulley 157. The second transmission cable 159 has one end connected to the first rotating member 151 and has the other end connected to the third rotating member 153. Specifically, the first transmission cable 158 and the second transmission cable 159 wrap around the first rotating member 151 in opposite directions and wrap around the third rotating member 153 in a same direction. In rotation of the first rotating member 151 or the third rotating member 153, the first movable pulley 156 is driven to move by the first transmission cable 158, and the second movable pulley 157 is driven to move by the second transmission cable 159.

The first transmission assembly further includes a first guide pulley 163. A portion of the first transmission cable 158 located between the first rotating member 151 and the first movable pulley 156 rides on the first guide pulley 163. Therefore, a portion of the first transmission cable 158 located between the first guide pulley 163 and the first movable pulley 156 is substantially parallel to another portion of the first transmission cable 158 located between the first movable pulley 156 and the third rotating member 153, and an absolute value of a linear speed of the portion of the first transmission cable 158 located between the first guide pulley 163 and the first movable pulley 156 is equal to an absolute value of a linear speed of the another portion of the first transmission cable 158 located between the first movable pulley 156 and the third rotating member 153. In other words, the linear speed of the portion of the first transmission cable 158 on one side of two opposite sides of the first movable pulley 156 and the linear speed of the another portion of the first transmission cable 158 on the other side of the two opposite sides of the first movable pulley 156 have a same absolute value. A portion of the second transmission cable 159 located between the first rotating member 151 and the second movable pulley 157 is substantially parallel to another portion of the second transmission cable 159 located between the second movable pulley 157 and the third rotating member 153, and substantially parallel to the another portion of the first transmission cable 158 located between the first movable pulley 156 and the third rotating member 153. In other words, a linear speed of the portion of the second transmission cable 159 located on one side of two opposite sides of the second movable pulley 157 and a linear speed of the another portion of the second transmission cable 159 located on the other side of the two opposite sides of the second movable pulley 157 have a same absolute value. The first guide pulley 163 may be a fixed pulley. It shall be understood that additional guide pulleys may be employed as needed to guide the first transmission cable 158 and the second transmission cable 159.

The first rotating member 151 is rotatable around a first central axis A1 of the first rotating member 151, to pull in one of the first cable 141 and the second cable 142 while releasing the other of the first cable 141 and the second cable 142 through the first transmission assembly, so as to achieve clockwise or counterclockwise rotation of the lower claw 135 around the second bolt 137 relative to the distal clevis 132. In an embodiment, the first shaft 161 is substantially parallel to the second shaft 162 and substantially parallel to the first central axis A1.

Specifically, during stationary of the third rotating member 153, when the driving motor controls the first rotating shaft 173 to drive the first rotating member 151 to rotate counterclockwise, the second transmission cable 159 is pulled in (i.e., wrapped around/wound on the first rotating member 151) and the first transmission cable 158 is released in equal lengths concurrently. In this way, the second cable 142 is pulled in via the second movable pulley 157 and the first cable 141 is released in equal lengths concurrently via the first movable pulley 156, thereby realizing clockwise rotation of the lower claw 135 around the second bolt 137 relative to the distal clevis 132 (referring to yaw 2 in FIG. 2). During stationary of the third rotating member 153, when the driving motor controls the first rotating shaft 173 to drive the first rotating member 151 to rotate clockwise, the first transmission cable 158 is pulled in (i.e., wrapped around/wound on the first rotating member 151) and the second transmission cable 159 is released in equal lengths concurrently. In this way, the first cable 141 is pulled in via the first movable pulley 156 and the second cable 142 is released in equal lengths via the second movable pulley 157 concurrently, thereby realizing counterclockwise rotation of the lower claw 135 around the second bolt 137 relative to the distal clevis 132.

With continued reference to FIGS. 3 and 4, the second transmission assembly is connected to the third cable 143 and the fourth cable 144, and further connected to the second rotating member 152 and the third rotating member 153. The second transmission assembly includes a third movable pulley 164, a fourth movable pulley 165, a third transmission cable 166, and a fourth transmission cable 167. The third movable pulley 164 is pivotably connected to a third pulley seat 183 via a third shaft 168, the third movable pulley 164 is pivotable around the third shaft 168, and the third cable 143 is connected to the third pulley seat 183. The fourth movable pulley 165 is pivotably connected to a fourth pulley seat 184 via a fourth shaft 169, the fourth movable pulley 165 is pivotable around the fourth shaft 169, and the fourth cable 144 is connected to the fourth pulley seat 184.

The third transmission cable 166 rides on the third movable pulley 164. The third transmission cable 166 has one end connected to the second rotating member 152 and has the other end connected to the third rotating member 153. The fourth transmission cable 167 rides on the fourth movable pulley 165. The fourth transmission cable 167 has one end connected to the second rotating member 152 and has the other end connected to the third rotating member 153. Specifically, the third transmission cable 166 and the fourth transmission cable 167 wraps around the second rotating member 152 in opposite directions, and a direction in which the third transmission cable 166 and the fourth transmission cable 167 wrap around the third rotating member 153 is opposite to a direction in which the first transmission cable 158 and the second transmission cable 159 wrap around the third rotating member 153. In other words, the third transmission cable 166 and the fourth transmission cable 167 wrap around the third rotating member 153 in a same direction. In rotation of the second rotating member 152 or the third rotating member 153, the third movable pulley 164 is driven to move by the third transmission cable 166, and the fourth movable pulley 165 is driven to move by the fourth transmission cable 167.

The second transmission assembly further includes a second guide pulley 171. A portion of the fourth transmission cable 167 located between the second rotating member 152 and the fourth movable pulley 165 rides on the second guide pulley 171. Therefore, a portion of the fourth transmission cable 167 located between the second guide pulley 171 and the fourth movable pulley 165 is substantially parallel to another portion of the fourth transmission cable 167 located between the fourth movable pulley 165 and the third rotating member 153. A portion of the third transmission cable 166 located between the second rotating member 152 and the third movable pulley 164 is substantially parallel to another portion of the third transmission cable 166 located between the third movable pulley 164 and the third rotating member 153, and substantially parallel to the another portion of the fourth transmission cable 167 located between the fourth movable pulley 165 and the third rotating member 153. The second guide pulley 171 may be a fixed pulley. It shall be understood that additional guide pulleys may be employed as needed to guide the third transmission cable 166 and the fourth transmission cable 167.

The second rotating member 152 is rotatable around a second central axis A2 of the second rotating member 152, to pull in one of the third cable 143 and the fourth cable 144 while releasing the other of the third cable 143 and the fourth cable 144 through the second transmission assembly, so as to achieve clockwise or counterclockwise rotation of the upper claw 134 around the second bolt 137 relative to the distal clevis 132. In an embodiment, the third shaft 168 is substantially parallel to the fourth shaft 169 and substantially parallel to the second central axis A2.

Specifically, during stationary of the third rotating member 153, when the driving motor controls the second rotating shaft 174 to drive the second rotating member 152 to rotate counterclockwise, the fourth transmission cable 167 is pulled in (i.e., wrapped around/wound on the second rotating member 152) and the third transmission cable 166 is released in equal lengths concurrently. In this way. the fourth cable 144 is pulled in via the fourth movable pulley 165 and the third cable 143 is released in equal lengths via the third movable pulley 164 concurrently, so that the upper claw 134 is rotated clockwise around the second bolt 137 relative to the distal clevis 132 (referring to yaw 1 in FIG. 2). During stationary of the third rotating member 153, when the driving motor controls the second rotating shaft 174 to drive the second rotating member 152 to rotate clockwise, the third transmission cable 166 is pulled in (i.e., wrapped around/wound on the second rotating member 152) and the fourth transmission cable 167 is released in equal lengths concurrently. In this way, the third cable 143 is pulled in via the third movable pulley 164 and the fourth cable 144 is released in equal lengths via the fourth movable pulley 165 concurrently, so that the upper claw 134 is rotated counterclockwise around the second bolt 137 relative to the distal clevis 132.

The rear-end transmission device 150 further includes a third guide pulley set 176 (referring to FIG. 4). The third guide pulley set 176 includes two fifth shafts 177 substantially parallel to each other, and each of the two fifth shafts 177 is provided with two third guide pulleys 178 pivotably disposed thereon. Each third guide pulley 178 may be a fixed pulley.

The four third guide pulleys 178 guide the first cable 141, the second cable 142, the third cable 143, and the fourth cable 144, respectively. Therefore, a portion of the first cable 141 located between the third guide pulley set 176 and the first movable pulley 156 is substantially parallel to the another portion of the first transmission cable 158 located between the first movable pulley 156 and the third rotating member 153. In addition, a portion of the second cable 142 located between the third guide pulley set 176 and the second movable pulley 157 is substantially parallel to the another portion of the second transmission cable 159 located between the second movable pulley 157 and the third rotating member 153. Furthermore, a portion of the third cable 143 located between the third guide pulley set 176 and the third movable pulley 164 is substantially parallel to the another portion of the third transmission cable 166 located between the third movable pulley 164 and the third rotating member 153. Furthermore, a portion of the fourth cable 144 located between the third guide pulley set 176 and the fourth movable pulley 165 is substantially parallel to the another portion of the fourth transmission cable 167 located between the fourth movable pulley 165 and the third rotating member 153. In this way, it is possible to achieve linear combination of driven of the first cable 141 and the second cable 142 by the first rotating member 151 and the third rotating member 153, and linear combination of driven of the third cable 143 and the fourth cable 144 by the second rotating member 152 and the third rotating member 153.

In this embodiment, by controlling rotation of the first rotating shaft 173 and the second rotating shaft 174, yaw and grip of the effector 133 can be realized.

Specifically, during stationary of the third rotating shaft 175, when both the first rotating shaft 173 and the second rotating shaft 174 rotate counterclockwise at equal angles, that is, when both the first rotating member 151 and the second rotating member 152 rotate counterclockwise at equal angles, both the upper claw 134 and the lower claw 135 rotate clockwise, thereby realizing yaw of the effector 133 in a direction (referring to FIG. 2). During stationary of the third rotating shaft 175, when both the first rotating shaft 173 and the second rotating shaft 174 rotate clockwise at equal angles, that is, when both the first rotating member 151 and the second rotating member 152 rotate clockwise at equal angles, both the upper claw 134 and the lower claw 135 rotate counterclockwise, thereby realizing yaw of the effector 133 in a reverse direction.

During stationary of the third rotating shaft 175, when the first rotating shaft 173 rotates counterclockwise and the second rotating shaft 174 rotates clockwise at equal angles concurrently, that is, when the first rotating member 151 rotates counterclockwise and the second rotating member 152 rotates clockwise at equal angles concurrently, the lower claw 135 rotates clockwise and the upper claw 134 rotates counterclockwise concurrently, to realize grip of the effector 133 (referring to FIG. 2). During stationary of the third rotating shaft 175, when the first rotating shaft 173 rotates clockwise and the second rotating shaft 174 rotates counterclockwise at equal angles concurrently, that is, when the first rotating member 151 rotates clockwise and the second rotating member 152 rotates counterclockwise at equal angles concurrently, the lower claw 135 rotates counterclockwise and the upper claw 134 rotates clockwise, to realize releasing of the grip of the effector 133.

The third rotating member 153 is rotatable around a third central axis A3 of the third rotating member 153 to perform, via the first transmission assembly and the second transmission assembly: pulling in at least one of the first cable 141 and the second cable 142 while releasing at least one of the third cable 143 and the fourth cable 144; or pulling in at least one of the third cable 143 and the fourth cable 144 while releasing at least one of the first cable 141 and the second cable 142, to achieve pitch of the wrist mechanism 130. In an embodiment, the first central axis A1 is substantially parallel to the second central axis A2 and substantially parallel to the third central axis A3.

In the present embodiment, during rotation of the third rotating member 153, the first cable 141 and the second cable 142 can be pulled in in equal lengths and the third cable 143 and the fourth cable 144 can be released in equal lengths concurrently via the first transmission assembly and the second transmission assembly, and during the rotation of the third rotating member 153, a total length of a portion of the first cable 141 and a portion of the second cable 142 that are pulled in is equal to a total length of a portion of the third cable 143 and a portion of the fourth cable 144 that are released, thereby achieving pitch of the wrist mechanism 130. Alternatively, during rotation of the third rotating member 153, the third cable 143 and the fourth cable 144 can be pulled in in equal lengths and the first cable 141 and the second cable 142 can be released in equal lengths concurrently, and during rotation of the third rotating member 153, a total length of a portion of the third cable 143 and a portion of the fourth cable 144 that are pulled in is equal to a total length of a portion of the first cable 141 and a portion of the second cable 142 that are released, thereby achieving pitch of the wrist mechanism 130.

Specifically, when the first rotating shaft 173 and the second rotating shaft 174 remain stationary and the driving motor controls the third rotating shaft 175 to drive the third rotating member 153 to rotate counterclockwise, the first transmission cable 158 and the second transmission cable 159 are released in equal lengths, and the third transmission cable 166 and the fourth transmission cable 167 are pulled in in equal lengths concurrently. That is, the first cable 141 and the second cable 142 are released in equal lengths and the third cable 143 and the fourth cable 144 are pulled in in equal lengths concurrently, thereby realizing the pitch of the wrist mechanism 130 in a direction. Similarly, when the first rotating shaft 173 and the second rotating shaft 174 remain stationary and the driving motor controls the third rotating shaft 175 to drive the third rotating member 153 to rotate clockwise, the first transmission cable 158 and the second transmission cable 159 are pulled in in equal lengths and the third transmission cable 166 and the fourth transmission cable 167 are released in equal lengths concurrently. That is, the first cable 141 and the second cable 142 are pulled in in equal lengths and the third cable 143 and the fourth cable 144 are released in equal lengths concurrently, thereby realizing the pitch of the wrist mechanism 130 in a reverse direction.

In the present embodiment, since rotation of the first rotating member 151, the second rotating member 152, and the third rotating member 153 are transmitted to the first cable 141, the second cable 142, the third cable 143, and the fourth cable 144 via the first movable pulley 156, the second movable pulley 157, the third movable pulley 164, and the fourth movable pulley 165, moving lengths (pulled-in or released lengths) of the first cable 141, the second cable 142, the third cable 143, and the fourth cable 144 are respectively half of moving lengths of the first transmission cable 158, the second transmission cable 159, the third transmission cable 166, and the fourth transmission cable 167, which means that a ratio of an input speed of the rear-end transmission device to an output speed of the rear-end transmission device is 2:1. Therefore, when there is a return difference in a reducer of the driving motor, or when there is a return difference at a connection between at least one of the first rotating shaft 173, the second rotating shaft 174, and the third rotating shaft 175 and the respective driving motors, the influence of the return difference on the wrist mechanism 130 at the front end is reduced by half, so that the motion accuracy of the wrist mechanism 130 may be improved.

According to an embodiment of the present disclosure, the rear-end transmission device is connected to four driving cables of the wrist mechanism, such that the wrist mechanism works cooperatively with the rear-end transmission device to achieve pitch, yaw, and grip of the wrist mechanism by pulling in or releasing the driving cables (i.e., the first cable, the second cable, the third cable, and the fourth cable) in equal lengths, which is simple in structure and accurate in transmission. In addition, a rotation angle of the rotating member is linearly related to a pitch angle, a yaw angle, or a grip angle of the wrist mechanism, and therefore, equal-length release and/or pulling-in of the driving cables can be ensured even when the above-mentioned angles of the wrist mechanism vary in a wide range.

Unless otherwise defined, technical and scientific terms used herein have the same meanings as are commonly understood by those skilled in the art of the present disclosure. Terms used herein are for specific practical purposes only and are not intended to limit the present disclosure. Terms such as “disposed/disposing” that appear herein may denote either a part is attached directly to another part or a part is attached to another part through middleware. Features described herein in an embodiment may be applied to another embodiment alone or in combination with other features unless the features are not applicable in the other embodiment or otherwise noted.

The present disclosure has been described by way of the above-described embodiments but it shall be understood that the above-described embodiments are for the purpose of illustrative and illustration only and are not intended to limit the present disclosure to the scope of the described embodiments. Those skilled in the art will appreciate that many more variations and modifications may be made according to the teachings of the present disclosure, all of which fall within the scope of the claims of the present disclosure.

What is claimed is:

1. A rear-end transmission device, comprising:
a first rotating member;
a second rotating member;
a third rotating member;
a first transmission assembly connected to a first cable and a second cable and further connected to the first rotating member and the third rotating member; and
a second transmission assembly connected to a third cable and a fourth cable and further connected to the second rotating member and the third rotating member;
wherein the first rotating member is rotatable to pull in one of the first cable and the second cable and to release another of the first cable and the second cable concurrently via the first transmission assembly;
the second rotating member is rotatable to pull in one of the third cable and the fourth cable and to release another of the third cable and the fourth cable concurrently via the second transmission assembly; and
the third rotating member is rotatable to perform via the first transmission assembly and the second transmission assembly:
pulling in at least one of the first cable and the second cable while releasing at least one of the third cable and the fourth cable; or
pulling in at least one of the third cable and the fourth cable while releasing at least one of the first cable and the second cable.

2. The rear-end transmission device of claim 1, wherein the first rotating member is rotatable around a first central axis of the first rotating member, the second rotating member is rotatable around a second central axis of the second rotating member, and the third rotating member is rotatable around a third central axis of the third rotating member; and
wherein the first central axis is substantially parallel to the second central axis and substantially parallel to the third central axis.

3. The rear-end transmission device of claim 2, wherein the first transmission assembly comprises:
a first movable pulley connected to the first cable;
a first transmission cable riding on the first movable pulley, wherein the first transmission cable has one end connected to the first rotating member and has another end connected to the third rotating member;
a second movable pulley connected to the second cable; and
a second transmission cable riding on the second movable pulley, wherein the second transmission cable has one end connected to the first rotating member and has another end connected to the third rotating member; wherein
the first transmission cable wraps around the first rotating member in a direction opposite to a direction in which the second transmission cable wraps around the first rotating member, and the first transmission cable wraps around the third rotating member in a direction same as a direction in which the second transmission cable wraps around the third rotating member; and
in rotation of the first rotating member or the third rotating member, the first movable pulley is driven to move by the first transmission cable and the second movable pulley is driven to move by the second transmission cable.

4. The rear-end transmission device of claim 3, wherein the first transmission assembly further comprises a first guide pulley, and wherein
a portion of the first transmission cable located between the first rotating member and the first movable pulley rides on the first guide pulley, such that a portion of the first transmission cable located between the first guide pulley and the first movable pulley is substantially parallel to a portion of the first transmission cable located between the first movable pulley and the third rotating member.

5. The rear-end transmission device of claim 4, wherein a portion of the second transmission cable located between the first rotating member and the second movable pulley is substantially parallel to a portion of the second transmission cable located between the second movable pulley and the third rotating member and substantially parallel to the portion of the first transmission cable located between the first movable pulley and the third rotating member.

6. The rear-end transmission device of claim 3, wherein the second transmission assembly comprises:
a third movable pulley connected to the third cable;
a third transmission cable riding on the third movable pulley, wherein the third transmission cable has one end connected to the second rotating member and has another end connected to the third rotating member;
a fourth movable pulley connected to the fourth cable; and
a fourth transmission cable riding on the fourth movable pulley, wherein the fourth transmission cable has one end connected to the second rotating member and has another end connected to the third rotating member; wherein
the third transmission cable wraps around the second rotating member in a direction opposite to a direction in which the fourth transmission cable wraps around the second rotating member, and each of the third transmission cable and the fourth transmission cable wraps around the third rotating member in a direction opposite to a direction in which each of the first transmission cable and the second transmission cable wraps around the third rotating member; and
in rotation of the second rotating member or the third rotating member, the third movable pulley is driven to move by the third transmission cable and the fourth movable pulley is driven to move by the fourth transmission cable.

7. The rear-end transmission device of claim 6, wherein
the first movable pulley is pivotably connected to a first pulley seat via a first shaft, and the first pulley seat is connected to the first cable, and wherein the second movable pulley is pivotably connected to a second pulley seat via a second shaft, and the second pulley seat is connected to the second cable, wherein the first shaft is substantially parallel to the second shaft and substantially parallel to the first central axis.

8. The rear-end transmission device of claim 6, wherein the third movable pulley is pivotably connected to a third pulley seat via a third shaft, and the third pulley seat is connected to the third cable, and wherein the fourth movable pulley is pivotably connected to a fourth pulley seat via a fourth shaft, and the fourth pulley seat is connected to the fourth cable, wherein the third shaft is substantially parallel to the fourth shaft and substantially parallel to the first central axis.

9. The rear-end transmission device of claim 6, wherein the second transmission assembly further includes a second guide pulley, and wherein
a portion of the fourth transmission cable located between the second rotating member and the fourth movable pulley rides on the second guide pulley, such that a portion of the fourth transmission cable located between the second guide pulley and the fourth movable pulley is substantially parallel to a portion of the fourth transmission cable located between the fourth movable pulley and the third rotating member.

10. The rear-end transmission device of claim 9, wherein a portion of the third transmission cable located between the second rotating member and the third movable pulley is substantially parallel to a portion of the third transmission cable located between the third movable pulley and the third rotating member, and substantially parallel to the portion of the fourth transmission cable located between the fourth movable pulley and the third rotating member.

11. A surgical instrument, comprising a rear-end transmission device, wherein the rear-end transmission device comprises:
a first rotating member;
a second rotating member;
a third rotating member;
a first transmission assembly connected to a first cable and a second cable and further connected to the first rotating member and the third rotating member; and
a second transmission assembly connected to a third cable and a fourth cable and further connected to the second rotating member and the third rotating member;
wherein the first rotating member is rotatable to pull in one of the first cable and the second cable and to release another of the first cable and the second cable concurrently via the first transmission assembly;
the second rotating member is rotatable to pull in one of the third cable and the fourth cable and to release another of the third cable and the fourth cable concurrently via the second transmission assembly; and
the third rotating member is rotatable to perform via the first transmission assembly and the second transmission assembly:
pulling in at least one of the first cable and the second cable while releasing at least one of the third cable and the fourth cable; or
pulling in at least one of the third cable and the fourth cable while releasing at least one of the first cable and the second cable.

12. The surgical instrument of claim 11, wherein the first rotating member is rotatable around a first central axis of the first rotating member, the second rotating member is rotatable around a second central axis of the second rotating member, and the third rotating member is rotatable around a third central axis of the third rotating member; and
wherein the first central axis is substantially parallel to the second central axis and substantially parallel to the third central axis.

13. The surgical instrument of claim 12, wherein the first transmission assembly comprises:
a first movable pulley connected to the first cable;
a first transmission cable riding on the first movable pulley, wherein the first transmission cable has one end connected to the first rotating member and has another end connected to the third rotating member;
a second movable pulley connected to the second cable; and
a second transmission cable riding on the second movable pulley, wherein the second transmission cable has one end connected to the first rotating member and has another end connected to the third rotating member; wherein
the first transmission cable wraps around the first rotating member in a direction opposite to a direction in which the second transmission cable wraps around the first rotating member, and the first transmission cable wraps around the third rotating member in a direction same as a direction in which the second transmission cable wraps around the third rotating member; and
in rotation of the first rotating member or the third rotating member, the first movable pulley is driven to move by the first transmission cable and the second movable pulley is driven to move by the second transmission cable.

14. The surgical instrument of claim 13, wherein the first transmission assembly further comprises a first guide pulley, and wherein
a portion of the first transmission cable located between the first rotating member and the first movable pulley rides on the first guide pulley, such that a portion of the first transmission cable located between the first guide pulley and the first movable pulley is substantially parallel to a portion of the first transmission cable located between the first movable pulley and the third rotating member.

15. The surgical instrument of claim 14, wherein a portion of the second transmission cable located between the first rotating member and the second movable pulley is substantially parallel to a portion of the second transmission cable located between the second movable pulley and the third rotating member and substantially parallel to the portion of the first transmission cable located between the first movable pulley and the third rotating member.

16. The surgical instrument of claim 13, wherein the second transmission assembly comprises:
a third movable pulley connected to the third cable;
a third transmission cable riding on the third movable pulley, wherein the third transmission cable has one end connected to the second rotating member and has another end connected to the third rotating member;
a fourth movable pulley connected to the fourth cable; and
a fourth transmission cable riding on the fourth movable pulley, wherein the fourth transmission cable has one end connected to the second rotating member and has another end connected to the third rotating member; wherein
the third transmission cable wraps around the second rotating member in a direction opposite to a direction in which the fourth transmission cable wraps around the second rotating member, and each of the third transmission cable and the fourth transmission cable wraps around the third rotating member in a direction opposite to a direction in which each of the first transmission cable and the second transmission cable wraps around the third rotating member; and
in rotation of the second rotating member or the third rotating member, the third movable pulley is driven to move by the third transmission cable and the fourth movable pulley is driven to move by the fourth transmission cable.

17. The surgical instrument of claim 16, wherein
the first movable pulley is pivotably connected to a first pulley seat via a first shaft, and the first pulley seat is connected to the first cable, and wherein the second movable pulley is pivotably connected to a second pulley seat via a second shaft, and the second pulley seat is connected to the second cable, wherein the first shaft is substantially parallel to the second shaft and substantially parallel to the first central axis.

18. The surgical instrument of claim 16, wherein the third movable pulley is pivotably connected to a third pulley seat via a third shaft, and the third pulley seat is connected to the third cable, and wherein the fourth movable pulley is pivotably connected to a fourth pulley seat via a fourth shaft, and the fourth pulley seat is connected to the fourth cable, wherein the third shaft is substantially parallel to the fourth shaft and substantially parallel to the first central axis.

19. The surgical instrument of claim 16, wherein the second transmission assembly further includes a second guide pulley, and wherein
a portion of the fourth transmission cable located between the second rotating member and the fourth movable pulley rides on the second guide pulley, such that a portion of the fourth transmission cable located between the second guide pulley and the fourth movable pulley is substantially parallel to a portion of the fourth transmission cable located between the fourth movable pulley and the third rotating member.

20. A surgical robot, comprising a surgical instrument, wherein the surgical instrument comprises a rear-end transmission device comprising:
a first rotating member;
a second rotating member;
a third rotating member;
a first transmission assembly connected to a first cable and a second cable and further connected to the first rotating member and the third rotating member; and
a second transmission assembly connected to a third cable and a fourth cable and further connected to the second rotating member and the third rotating member;
wherein the first rotating member is rotatable to pull in one of the first cable and the second cable and to release another of the first cable and the second cable concurrently via the first transmission assembly;
the second rotating member is rotatable to pull in one of the third cable and the fourth cable and to release another of the third cable and the fourth cable concurrently via the second transmission assembly; and
the third rotating member is rotatable to perform via the first transmission assembly and the second transmission assembly:
pulling in at least one of the first cable and the second cable while releasing at least one of the third cable and the fourth cable; or
pulling in at least one of the third cable and the fourth cable while releasing at least one of the first cable and the second cable.
